(19) United States
(12) Patent Application Publication
(10) Pub. No.: US 2005/0107455 A1
(43) Pub. Date: May 19, 2005

(54) Novel tricycloimidazoline derivatives method for production and use thereof as medicaments

(76) Inventors: Bernard Vacher, Castres (FR); Bernard Bonnaud, Lagarrigue (FR); Marc Marien, Castres (FR); Thierry Imbert, Viviers-Les-Montagnes (FR)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 10/504,642

(22) PCT Filed: Feb. 14, 2003
(86) PCT No.: PCT/FR03/00480

(30) Foreign Application Priority Data
Feb. 14, 2002 (FR) 02/01839

(51) Int. Cl. C07D 233/08; A61K 31/4164

(52) U.S. Cl. 514/401; 548/347.1

(57) ABSTRACT

The invention relates to compounds of general formula (1) in which R1=H, F or OCH₃ and may occupy the 2, 3, 4 or 5 position on the aromatic carbocycle, R2=H or CH₃, R3=H, CH₃, OH or OCH₃, R4=H and R3 and R4 together can be a carbonyl group (C=0), the addition salts and optionally the hydrates of additional salts with pharmaceutically-acceptable mineral or organic acids and the isomers and tautomers thereof.

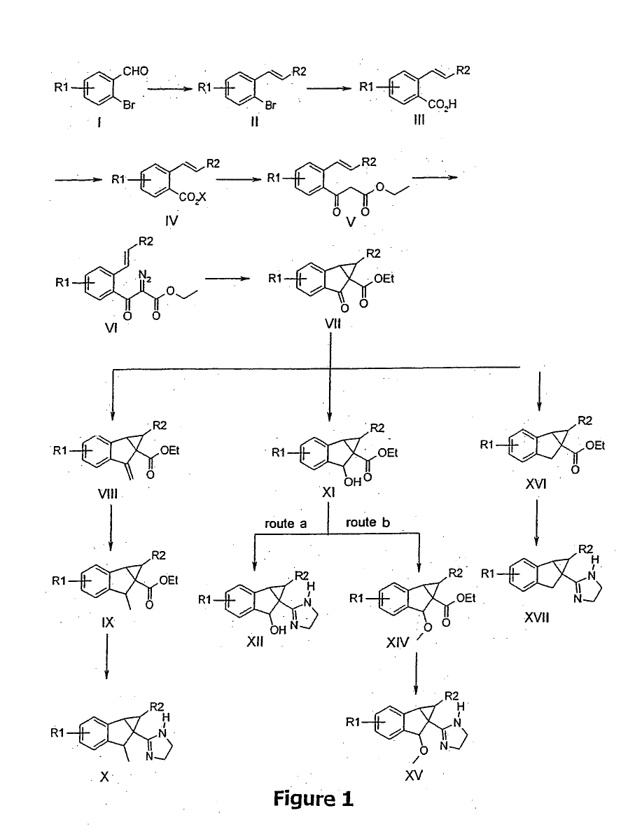

The present invention relates to novel tricyclic derivatives substituted with a 4,5-dihydro-1H-imidazole group. The compounds of the invention interact selectively with presynaptic and/or postsynaptic alpha-2 type adrenergic receptors (J. Neurochem. 2001, 78, 685-93), on which they behave like partial agonists, antagonists or inverse agonists. As such, the compounds of the invention are therefore potentially useful in the treatment of pathologies or conditions sensitive to an adrenergic regulation controlled by the adrenergic alpha-2 receptors. The list of pathologies considered as being sensitive to such regulation is excessively long. However, the field of application of the present invention is limited to the treatment of neurodegenerative diseases and also to the treatment of the evolution of said diseases (Psychopharmacology 1996, 123(3), -239-49; Prog. Neuro-Psychopharmacol. Biol. Psychiatry, 1999, 23(7), 1237-46; FR 2 789 681; WO 98/35670; WO-98/06393; WO 95/00145; WO 94/13285), in particular to the treatment of Alzheimer's disease or to the treatment of the, evolution of Alzheimer's disease (U.S. Pat. No. 5,281,607; FR 2 795 727; WO 95/01791; WO 94/15603).

Alzheimer's disease is the progressive degenerative disease that is most widespread in the elderly population. It is estimated that more than: 15 million people are affected (New Engl. J. Med. 1999, 341(22), 1670-79; Drug Benefit Trends 2001, 13/7, 27-40). At the present time, acetylcholinesterase inhibitors (e.g. tacrine, donepezil, rivastigmine and galantamine) constitute the main therapeutic strategy. However, this therapeutic approach is purely symptomatic and therapeutic benefits obtained are, to say the most, modest (Drugs 2001, 61/1, 41-52). Since effective therapeutic options against Alzheimer's disease are limited (Curr. Opin. Invest. Drugs 2001, 2(5), 654-56), the discovery of novel treatments involving molecules endowed with a mechanism of action different than that of the molecules currently clinically available and capable of treating or delaying the evolution of the disease is thus highly desirable.

It has been shown, in vitro and in animals, that a substance that activates the noradrenergic system can counter the progress of neurone degeneration (J. Neuro-physiol. 1998, 79(6), 2941-63; Pharmacol. Biochem. Behav. 1997, 56(4), 649-55; J. Cereb. Blood Flow Metabolism 1990, 10(6), 885-94) and, furthermore, has the property of stimulating neuronal growth (J. Comp. Neurol. 1974, 155(1), 15-42; Neuroscience 1979, 4(11), 1569-82; Neuroreport 1991, 2, 528-8). It follows that compounds with antagonist or inverse agonist properties on the adrenergic alpha-2 receptors, in particular on the presynaptic alpha-2 receptors, may be useful in the treatment of neurodegenerative diseases. Given the therapeutic potential of compounds endowed with antagonist or inverse agonist, activity for the adrenergic alpha-2 receptors, the discovery of novel structures endowed with such properties is highly desirable. In this respect, the Applicant has discovered that tricyclic derivatives substituted with a 4,5-dihydro-1H-imidazole group interact selectively with the adrenergic receptors of the alpha-2 subtype, on which they behave like antagonists or inverse agonists.

Many presynaptic and/or postsynaptic antagonists and/or partial antagonists of the adrenergic alpha-2 receptors are known and described in the literature. Although the compounds under consideration belong to different chemical classes (Idrugs 2001, 4(6), 662-76), some comprise in their chemical structure a common unit of the 4,5-dihydro-1H-imidazole type. Among these compounds, examples that may be mentioned include compounds of the type:
dihydroisoindolemethyl- (EP 275 639; EP 313 288; EP 413 433; U.S. Pat. No. 4,959,374; WO 93/09113 and Eur. J. Pharmacol. 1989, 168(3), 381-6);
2,3-dihydroindole- (FR 2 577 223; U.S. Pat. No. 4,908,376 and U.S. Pat. No. 4,912,125);
5,6-dihydrothieno[3,4-c]pyrrolmethyl- (EP 682 028);
1,2,4,5-tetrahydro-1H-pyrrolo[3,2,1]indole- (FR 2 583 048; FR 2 611 717; DE 4 325 491 and Life Sci. 1998, 62(9), 839-52);
1,2,3,4-tetrahydroisoquinolinemethyl- (J. Med. Chem. 1990, 33(2), 596-600);
1,2,3,4-tetrahydroquinoline- (U.S. Pat. No. 5,017,584; JP 60-058 976 and FR 2 576 308);
2,3-dihydrobenzochromene- (Heterocycles 2001, 55(2), 387-92);
1,4-benzodioxane- (EP 33 655; EP 58 006; EP 92 328; Neurochem. Int. 1996, 30(1), 47-53 and Mol. Neuropharmacol. 1992, 1(4), 219-24);
1,4-benzodioxanemethyl- (EP 74 711);
4H-1,3-benzodioxine- (J. Pharmacol. Exp. Ther. 1995, 272(2), 681-8);
2,3-dihydro-1,4-benzoxathiine- (Eur. J. Med. Chem. 1987, 22(4), 273-6);
2H-1-benzopyrane- and naphthalene- and 3,4-dihydronaphthalene- (Bioorg. Med. Chem. 1997, 5(5), 843-56);
dibenzo[1,4]dioxepine- and dibenzopyrane- (Eur. J. Med. Chem. 1991, 26(2) 207-13);
2,3-dihydro-2-benzofuran- (U.S. Pat. No. 4,411,908 and WO-92/05171);
furopyridin-efaroxan analogs (Heterocycles 1998, 48(12), 2529-34);
2-benzofuran- (Eur. J. Pharmacol. 1996, 304(1-3), 221-29 and Eur. J. Pharmacol. 1998, 353(1), 123-35);
1-hydroxy-2-phenoxy-2-phenylethyl- (Eur. J. Med. Chem. 1990, 25(9), 757-63;
phenoxyphenylethyl- (EP 423 802);
cirazoline analogs (Bioorg. Med. Chem. 2000, 8(5), 883-88);
phentolamine analogs (Med. Chem. Res. 1997, 7(1), 53-65);
medetodimine analogs (J. Med. Chem. 1992, 35(4), 750-5);
2-hydroxyindane- (Bioorg. Med. Chem. 2000, 8(8), 1861-69);
2,3-dihydro-1H-indene (FR 2 542 738 and J. Med. Chem. 1988, 31(5), 944-48);
3,4-dihydro-2-naphthalenemethyl- and 2-naphthalenemethyl- and 2H-1-benzopyranemethyl- and benzofuranmethyl- and indenemethyl- and indanemethyl- (EP 1 010 693);
quinoxaline- (ES 2 009 246);
benzospiroalkene- (EP 0 635 497);
bicyclo[4,2,0]-1,3,5-octatriene- (U.S. Pat. No. 4,567,181).

It is also noteworthy that some of the compounds mentioned above have only relatively minor structural differences. Publication J. Med. Chem. 2001, 44(5), 787-805 describes compounds whose carbon skeleton is of the 1a,2,3,3a,7a,7b-hexahydro-1H-cyclopropa[a]-naphthalene type (figure a): 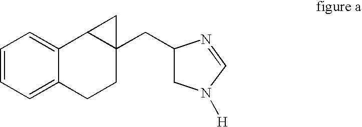
the carbon-based polycycle is linked to a heterocycle of the 4-(4,5-dihydro-1H-imidazole) type via a mono-methylene (CH₂) bridge. All the compounds of the invention-have a carbon-based skeleton of the 1a,6-dihydro-1H-cyclopropa[a]indene type directly linked to a heterocycle of the 2-(4,5-dihydro-1H-imidazole) type. These structural differences (i.e. size of the carbon-based polycyclic system, number of coupling members in the carbocycle-heterocycle junction, isomerism of the nitrogen heterocycles) induce significant differences in their pharmacological profiles. For example, under ordinary temperature conditions, the conformational mobility inherent to the structure of the compounds represented by figure a is very much higher than that of the compounds of the invention. This results in different activity profiles. Thus, for example, the compounds of the invention interact selectively with the adrenergic alpha-2 receptors, whereas the compounds described in J. Med. Chem. 2001, 44(5), 787-805 also interact with the serotonin uptake sites.

The closest state of the art is represented by compounds of the polycyclic indanylimidazole type (WO 01/85698) corresponding to the following formula (figure b): 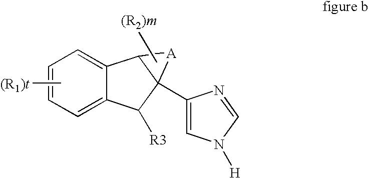
in which, inter alia:
A can form, with the two carbon atoms to which it is attached, a 3-membered carbon-based monocycle;
m may be 0 or 1;
R2 may be a (C₁₋₆)alkyl group;
t may be 0 or 1;
t is 1 and R1 may be a halogen or a (C₁₋₆) alkyloxy group;
R3 may be a hydrogen, OH, ═O, (C₁₋₆)alkyl or (C₁₋₆)alkyloxy;
   the compounds represented above and the compounds of the present invention thus differing in, the nature of their nitrogen heterocycle. In this respect, it is known to a person skilled in the art familiar with the field that the nature of the nitrogen heterocycle constitutes one of the major determining factors of the pharmacological activity profile of the ligand. Given the large number of structures involving a 4,5-dihydro-1H-imidazole unit, which are already known for their adrenergic alpha-2 properties, it is surprising that the 2-(1a,6-dihydro-1H-cyclopropa[a]inden-6a-yl)-4,5-dihydro-1H-imidazole unit gives the compounds of the invention a quite unique pharmacological profile. Specifically, in vitro, the following is shown:
selectivity of the compounds of the invention for the human alpha-2A versus human alpha-2B subtype. This characteristic is important since the intended indication involves a central action of the product in cerebral structures in which the alpha-2B sites do not appear to be preferentially distributed (Eur. J. Pharmacol. 1999, 366(1), 35-0.39; Nature 1999, 402, 181-83). An interaction with the alpha-2B subtypes thus tends to increase above all the probability of appearance of adverse effects;
intrinsic activity of the inverse agonist type of the compounds of the invention on the alpha-2A receptors, which is thus different than that of the compounds claimed in WO 01/85698.

Furthermore, it is shown, in vivo, that the products of the invention are capable of countering the effect of scopolamine in a test of memory deficit considered as a representative animal model of the memory disorders that are manifested during Alzheimer's disease (Psychopharmacology 1992, 106, 26-30; Exp. Neurol. 2000, 163, 495-529). The compounds of the invention, endowed with such an activity profile, are therefore potentially useful for treating diseases or disorders that are sensitive to the action of partial: agonists, antagonists or inverse agonists of the adrenergic alpha-2 receptors, for instance neurodegenerative diseases for which there is a strong therapeutic need.

Finally, the process for preparing the compounds of the invention is different than that for the compounds claimed in WO 01/85698 and involves novel reaction intermediates.

More specifically, a subject of the present invention is novel 2-(1a,6-dihydro-1H-cyclopropa[a]inden-6a-yl)-4,5-dihydro-1H-imidazole derivatives, which, in base form, correspond to the general formula (1): 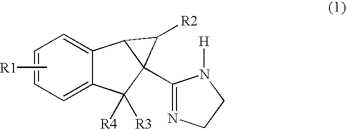
in which:
R1 represents a hydrogen atom, a fluorine atom or a methoxyl (OCH₃) group. The substituent R1 on the aromatic carbocycle may be in position 2, 3, 4 or 5;
R2 represents a hydrogen atom or a methyl group;
R3 represents a hydrogen atom, a methyl group, a hydroxyl group. (OH) or a methoxyl group (OCH₃);
R4 is a hydrogen atom;
R3 and R4 together represent a carbonyl group (C═O); the addition salts thereof and optionally the hydrates of the addition salts with pharmaceutically acceptable mineral acids or organic acids, and also the tautomeric forms, thereof, the enantiomers- and mixtures of enantiomers and the stereoisomers in pure form or in the form of a racemic or non-racemic mixture.

In one particular embodiment of the invention, the compounds of formula (1) in which:
R1 and R2 have the same meaning as above;
R3 represents a methyl group, a hydroxyl group (OH) or a methoxyl group (OCH₃);
R4 is a hydrogen atom;
the substituents R3 and 4,5-dihydro-1H-imidazole are in anti-periplanar positions relative to the plane defined by the indane ring system;
   are in all cases the preferred stereoisomers of the products of the invention.

In another particular embodiment of the invention, the compounds of formula (1) in which:
R1 has the same meaning as above;
R2 represents a methyl group;
R3 represents a hydrogen atom, a methyl group, a hydroxyl group (OH) or a methoxyl group (OCH₃);
R4 is a hydrogen atom;
R3 and R4 together represent a carbonyl group (C═O);
the substituents R2 and 4,5-dihydro-1H-imidazole are in syn-periplanar positions relative to the plane defined by the cyclopropane ring;
   are in all cases the preferred stereoisomers of the products of the invention.

By the term “anti-periplanar” the inventors mean the relative configurations of the molecules (1) for which the substituents R3 and 4,5-dihydroimidazole are on either side of the plane defined by the indane ring system. By the term “syn-periplanar”, the inventors mean the relative, configurations of the molecules (1) for which the substituents R2 and 4,5-dihydroimidazole are on the same side of the plane defined by the cyclopropane ring.

The compounds of general formula. (1) may exist in several tautomeric forms. Although not explicitly reported in the present patent application, to simplify the graphic representation of the structural formulae, such tautomeric forms are nevertheless included in the field of application of the invention. The compounds of the invention comprise several asymmetric carbon atoms in their structure.

As a result, they exist in the form of enantiomers and diastereoisomers. The invention relates not only to each pure stereoisomer, i.e. combined with less than 5% of another stereoisomer or of a mixture of other stereoisomers, but also to the mixture of one or more stereoisomers in all proportions. The compounds of the invention may thus intervene as pure stereoisomers or racemic or non-racemic mixtures of stereoisomers.

Finally, the invention covers the process for preparing the derivatives of general formula (1).

The derivatives of general formula (1) may be obtained by the process described in the scheme illustrated in appendix 1. The preparation of the compounds of the invention uses as starting material suitable 2-bromo-benzaldehydes, of formula (1), which are commercially available or known in the literature (i.e. RN 6630-33-7; RN 10401-18-0; RN 43192-31-0; RN 7507-80-0; RN 126712-07-0; RN 59142-68-6; RN 94569-84-3; RN 360575-28-6 or prepared by reduction of the corresponding acid RN 132715-69′-6). The derivatives of the 2-ethenyl type of formula (II-1) are obtained via a Wittig reaction performed using methyltriphenylphosphonium bromide in basic medium. The derivatives of the 2-(1-propenyl)-type of formula (II-2) and of (E) stereochemistry are prepared selectively in 2 steps according to the method described in Tetrahedron 1995, 51(37), 10115-24: addition of ethylmagnesium bromide to the aldehyde function followed by a dehydration reaction of the secondary alcohol obtained in acidic medium. The introduction of the carboxylic acid function onto the derivatives of formula (II) is performed by means of a bromine-lithium exchange reaction followed by trapping the organolithium reagent formed using CO₂. The 2-ethenylbenzoic acids (III-1) and (E)-2-(1-propenyl)benzoic acid (III-2) are compounds that are known in the literature (RN 27326-43-8 and RN 68692-67-1, respectively). The derivatives of formula (III), activated either in acyl chloride form or in amide form (IV), Synlett 1994, 2, 105-6, are converted into β-keto esters (V) by applying a method similar to that described in Synthesis 1993, 3, 290-92. The key intermediate in the preparation of the compounds of the invention is the 6-oxo-1a,6-dihydro-1H-cyclopropa[a]indene-6a-carboxylic acid ester of formula (VII). This ester is obtained by intramolecular addition of a carbenoid onto the double bond according to Doyle, M. P.; McKervey, M. A.; Ye, T. Modern Catalytic Methods for Organic Synthesis with Diazo Compounds, John Wiley & Sons, Inc. 1998, chapter 5, pages 238-288. The carbenoid is obtained by decomposition of a precursor of the diazo type (VI), which is itself prepared from the 3-oxo-3-(2-enyl-aryl)propionic acid esters of formula (V) according to the method described in Synth. Commun. 1987, 17(4), 1709-16.

Starting with the compound of formula (VII), all the compounds of the invention are prepared. Thus, the compounds (X) in which: R1 is H, F or OCH₃; R2 is H or CH₃; R3 is CH₃ and R₄ is H are obtained by: methylenation of (VII) using methyltriphenylphosphonium bromide in the presence of a base according to a standard Wittig reaction; reduction of the exocyclic double bond formed using the diimide according to a procedure similar to that described in Tetrahedron 1976, 32, 2157-62 and condensation of ethylenediamine in the presence of trimethylaluminum onto the esters (IX) according to a technique described in J. Org. Chem. 1987, 46, 2824-26.

The compounds of formulae (XII, XIII and XV) in which: R1 is H, F or OCH₃; R2 is H or CH₃; R3 is OH, OCH₃; R4 is H or R3 and R4 together form a carbonyl group (═O) are, or their part, prepared from the intermediate (VII). Thus, the 6-oxo function of (VII) may be reduced to the alcohol (XI) by means, for example, of a hydride donor. The reduction with sodium borohydride in cold ethanol is diastereoselective; the isomer in which the hydroxyl (OH) and cyclopropane groups are in syn-periplanar positions relative to the plane defined by the indane ring system is the only one observed in the reaction under consideration. The secondary alcohol (XI) may, then be either directly converted into the expected heterocycle (XII), route a, appendix 1; or methylated to the ether (XIV) and then converted into the expected heterocycle (XV), route b, appendix 0.1. Oxidation of the alcohols of formula (XII) gives the compounds of formula (XIII).

The compounds of formula (XVI) in which: R1 is H, F or OCH₃; R2 is H or CH₃; R3 and R4 are H, are derived from the total reduction of the 6-oxo function of the compound of formula (VII) according to a method similar to that described in J. Org. Chem. 1973, 38(15), 2675-81. The nitrogen heterocycle contained in (XVII) is then formed as described previously.

The compounds of formulae (X), (XII), (XIII), (XV) and (XVII) constitute all the compounds of the invention.

A subject of the invention is also pharmaceutical compositions containing, as active principle, at least one of the derivatives of general formula (1) or a salt thereof or hydrates of salts thereof in combination with one or more inert supports or other pharmaceutically acceptable vehicles.

The pharmaceutical compositions according to the invention may be, for example, compositions for oral, nasal, sublingual, rectal or parenteral administration. As examples of compositions for oral administration, mention may be made of tablets, gel capsules, granules, powders and oral solutions or suspensions.

The formulations that are suitable for the chosen administration form are known and described, for example, in: Remington, The Science and Practice of Pharmacy, 19th edition, 1995, Mack Publishing Company.

The effective dose of a compound of the invention varies as a function of numerous parameters, for instance the chosen route of administration, the weight, age, sex, degree of advancement of the pathology to be treated and sensitivity of the individual to be treated. Consequently, the optimum dosage will have to be determined, as a function of the parameters considered pertinent, by the specialist in the art. Although the effective doses of a compound of the invention can vary within large proportions, the daily doses may range between 0.01 mg and 100 mg per kg of body weight of the individual to be treated. A daily dose of a compound of the invention of between 0.10 mg and 50 mg per kg of body weight of the individual to be treated is, however, preferred.

The pharmaceutical compositions according to the invention, are useful in the treatment of neurodegenerative diseases.

### EXAMPLES

The examples that follow illustrate the invention but do not limit it in any way.

In the examples and the reference examples below:
(i) the reaction progress is monitored by thin layer chromatography (TLC) and consequently the reaction times are mentioned only as a guide;
(ii) different crystalline forms may give different melting points; the melting points reported in the present patent application are those of the products prepared according to the method described and are uncorrected;
(iii) the structure of the products obtained according to the invention is confirmed by the nuclear magnetic resonance (NMR) and infrared (IR) spectra and the elemental analysis, the purity of the final products is checked by TLC, and the enantiomeric purity of the reaction intermediates and final products is determined by chiral-phase HPLC;
(iv) the NMR spectra are recorded in the solvent indicated. The chemical shifts (δ) are expressed in parts per million (ppm) relative to tetra-methylsilane. The multiplicity of the signals is indicated by: s, singlet; d, doublet; t, triplet; q, quartet; m, multiplet; b, broad;
(v) the various symbols of the units have their usual meaning: μg (microgram); mg (milligram); g (gram); ml (milliliter); mV (millivolt); ° C. (degrees Celsius); mmol (millimole); nmol (nanomole); cm (centimeter); nm (nanometer); min (minute); ms (millisecond), Hz (hertz); [α] (specific optical rotation measured at 5.89 nm, 25° C. and at a concentration c, in the present invention the dimension deg cm² g⁻¹ is always implied); the pressures are given in millibar (mb);
(vi) the abbreviations have the following meanings: mp (melting point); bp (boiling point); AUC (area under the curve);
(vii) the term “room temperature” means a temperature of between 20° C. and 25° C.

### Example 1

### Ethyl 6-oxo-1a,6-dihydro-1H-cyclopropa[a]-—indene-6a-carboxylate (VII-1)

A solution of 23.06 ml (0.153 mol) of DBU in 35 ml of anhydrous THF is added dropwise to a solution of 33.42 g (0.153 mol) of ethyl ortho-vinylbenzoylacetate (V-1), 36.76 g (0.153 mol) of para-acetamidobenzenesulfonyl azide and 300 ml of anhydrous THF, while stirring on an ice bath and under nitrogen. After stirring for 16 hours at room temperature, the garnet-red solution is poured into a mixture of saturated aqueous NH₄Cl solution and ice. The mixture is extracted twice with ethyl acetate. The organic phases are separated out by settling and washed with water and then with brine. After drying over MgSO₄ and filtration, the solvent is evaporated off under vacuum (temperature below 40° C.). The crystalline mass obtained is taken up in a 50/50 cyclohexane/ethyl acetate mixture and the para-acetamidobenzenesulfonylamide is filtered off. The mother liquors are evaporated to dryness under vacuum (T°<40° C.). The brown oil obtained is purified by rapid filtration on 200 g of silica using CH₂Cl₂ as solvent. After removal of the solvent (T°<40° C.), 35.81 g (95.8%) of an orange-colored oil are obtained, and are used directly in the cycloaddition reaction.

The solution of the diazoacetate (VI-1) obtained previously in 50 ml of anhydrous CH₂Cl₂ is introduced dropwise to a suspension of 1.18 g of rhodium acetate and 250 ml of anhydrous CH₂Cl₂, with stirring at room temperature. After stirring overnight at room temperature, the catalyst is filtered off and the solvent is removed under vacuum. The crude product is purified by chromatography on 320 g of silica, using cyclohexane containing 10% ethyl acetate as solvent.

The title product is obtained (23.9 g);

Yield: 75.7%

C₁₃H₁₂O₃: 216.24

IR (film) ν: 1720 and 1746 cm¹ (C═O)

¹H NMR (CDCl₃): 1.33 (t, 3H) 1.744 (t, 1H); 2.39 (dd, 1H); 3.37 (dd, 1H); 4.29 (q, 2H); 7.35 (t, 1H); 7.45 (d, 1H); 7.51 (t, 1H); 7.70 (d, 1H)

¹³C NMR (CDCl₃): 14.13; 32.04; 38.61; 39.77; 61.56; 124.43; 125.31; 127.63; 134.11; 134.13; 151.46; 168.50; 195.44.

### Example 2

### Ethyl 1a,6-dihydro-1H-cyclopropa[a]indene-6a-carboxylate (XVI-1)

1.7 ml (10.64 mmol) of triethylsilane are added dropwise to a solution of 0.92 g (4.25 mmol) of (VII-1) in 4 ml of trifluoroacetic acid, with stirring, under nitrogen and on an ice bath. After stirring for 4 hours at room temperature, the solution is poured into an ice/water mixture. After addition of ethyl acetate, the mixture is basified by addition of sodium bicarbonate with vigorous stirring. The organic phase is separated out by settling and then washed with water and then with brine. After drying over MgSO₄ and filtration, the brown oil obtained is purified by chromatography on silica, using cyclohexane containing 2% ethyl acetate as eluent. The title product is obtained (0.47 g);

Yield: 54.6%

C₁₃H₁₄O₂: 202.24

IR (film) ν: 1721 cm¹ (C═O)

¹H NMR (CDCl₃): 0.68 (t, 1H); 1.27 (t, 3H); 1.98 (dd, 1H); 2.95 (ddd, 1H); 3.06 (d, 1H); 3.72 (d, 1H); 4.18 (q, 2H); 7.13 (m, 2H); 7.18 (m, 1H); 7.27 (m, 1H).

### Example 3

### Ethyl 1-exo-methyl-1a,6-dihydro-1H-cyclopropa[a]indene-6a-carboxylate (XVI-2)

C₁₄H₁₆O₂: 216.28

IR (film) ν: 1717 cm⁻¹ (C═O)

¹H NMR (CDCl₃): 1.04 (m, 1H); 1.29 (t, 3H); 1.35 (d, 3H); 2.77 (d, 1H, J=4.4 Hz); 3.14 (d, 1H); 3.58 (d, 1H); 4.21 (m, 2H); 7.11 (m, 3H); 7.24 (m, 1H).

### Example 4

### Ethyl 6-methylene-1a,6-dihydro-1H-cyclopropa[a]indene-6a-carboxylate (VIII)

2.56 g (21 mmol) of KOtBu are added portionwise to a suspension of 7.5 g (21 mmol) of methyltriphenylphosphonium bromide and 45 ml of anhydrous THF with stirring at room temperature. The suspension is stirred for 1 hour 30 minutes, and a solution of 3.02 g (14 mmol) of (VII-1) in 5 ml of anhydrous THF is then introduced dropwise, with cooling over an ice bath. After stirring overnight at room temperature, the suspension is poured into saturated aqueous NH₄Cl solution and extracted twice with ethyl acetate. The organic phases are washed with water and then with brine. After drying over MgSO₄ and filtration, the solvent is removed under vacuum. The triphenylphosphine oxide is crystallized from isopropyl ether and the mother liquors are evaporated to dryness under vacuum. The residual oil is purified by chromatography on silica, using cyclohexane containing 3% ethyl acetate as eluent. The title product is obtained (1.8 g);

Yield: 59%

C₁₄H₁₄O₂: 214.25

¹H NMR (CDCl₃): 1.04 (t, 1H); 1.30 (t, 3H); 2.16 (dd, 1H); 3.16 (dd, 1H); 4.21 (m, 2H); 5.7.3 (s, 1H); 5.84 (s, 1H); 7.19 (m, 2H); 7.30 (m, 1H); 7.47 (m, 1H).

### Example 5

### Ethyl 6-methyl-1a,6-dihydro-1H-cyclopropa-[a]indene-6a-carboxylate (IX)

3.65 ml (26 mmol) of triethylamine are added dropwise to a suspension of 2.8 g (13 mmol) of (VIII), 5.6 g (26 mmol) of 2,4,6-trimethylbenzenesulfonyl hydrazide and 20 ml of anhydrous methanol, with stirring at room temperature, and the mixture is then refluxed for 5 hours. The methanol is removed under vacuum; saturated aqueous 10% NaHCO₃ solution is added and the mixture is extracted twice with ether. The organic phases are washed with brine, dried over MgSO₄, filtered and evaporated to dryness under vacuum. The residual oil is purified by chromatography on silica, using cyclohexane containing 50% dichloromethane as eluent. The title product is obtained (2.19 g);

Yield: 78%

C₁₄H₁₆O₂: 216.27

¹H NMR (CDCl₃): 0.64 (t, 1H); 1.27 (t, 3H); 1.36 (d, 3H); 1.77 (dd, 1H); 2.93 (dd, 1H); 4.04 (q, 1H); 4.19 (q, 2H); 7.14 (m, 3H); 7.25 (d, 1H).

### Example 6

### Ethyl 6-hydroxy-1a,6-dihydro-1H-cyclopropa-[a]indene-6a-carboxylate (XI-1)

1.97 g (36.4 mmol) of KBH₄ are added portionwise to a solution of 5.25 g (24.3 mmol) of (VII-1) in 50 ml of absolute ethanol, with stirring on an ice bath, the mixture is then stirred overnight while allowing the bath to return to room temperature. The ethanol is removed under vacuum and the residue is taken up in an ice/water mixture and extracted twice with ethyl acetate. The organic phases are washed with brine, dried over MgSO₄, filtered and evaporated to dryness under vacuum. The residue is purified by chromatography on silica, using cyclohexane containing 20% ethyl acetate as eluent. The title compound is obtained (4.98 g);

Yield: 95%

C₁₃H₁₄O₃: 218.24

IR (film) ν: 3440 cm⁻¹ (OH); 1720 cm⁻¹ (C═O)

¹H NMR (CDCl₃): 1.67 (t, 1H); 1.29 (t, 3H); 1.83 (dd, 1H); 2.34 (d, 1H); 3.01 (dd, 1H); 4.22 (m, 2H); 6.03 (d, 1H); 7.22 (m, 3H); 7.31 (m, 1H).

### Example 7

### Ethyl 6-methoxy-1a,6-dihydro-1H-cyclopropa-[a]indene-6a-carboxylate (XIV-1) 26.53 g (114.5 mmol) of Ag₂O are added to a solution of

5 g (22.9 mmol) of (XI-1) and 80 ml of anhydrous acetonitrile, with stirring at room-temperature, followed by rapid dropwise addition of 7.13 ml (114.5 mmol) of methyl iodide. The suspension is stirred at room temperature, in the absence of light, for 48 hours. The suspension is filtered and the solvent is removed under vacuum. The residual oil is purified by chromatography on silica, using cyclohexane containing 5% ethyl acetate as eluent. The title compound is obtained (4.3 g);

Yield: 81%

C₁₄H₁₆O₃: 232.27

¹H NMR (CDCl₃): 1.29 (m, 4H); 1.92 (dd, 1H); 2.79 (dd, 1H); 3.57 (s, 3H); 4.21 (m, 2H); 5.73 (s, 1H); 7.20 (m, 3H); 7.28 (m, 1H).

### Example 8

### 2-(1a,6-Dihydro-1H-cyclopropa [a]inden-6a-yl)-4,5-dihydro-1H-imidazole; (XVII-1)

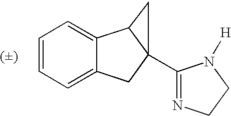

0.23 ml (3.45 mmol) of ethylenediamine is introduced dropwise into a solution of 1.5 ml (3 mmol) of a 2M solution of trimethylaluminum in toluene and 10 ml of anhydrous toluene, with vigorous stirring at −10° C. Stirring is continued at room temperature for 30 minutes, followed by dropwise addition of a solution of 0.47 g (2.3 mmol) of (XVI-1) in 2 ml of anhydrous toluene. The mixture is refluxed for 2 hours. 1.3 ml of water are added slowly over an ice bath with vigorous stirring, and the mixture is kept at room temperature for 30 minutes. The organic phase is separated out by settling, diluted with ethyl acetate, washed with brine, dried (Na₂SO₄) and evaporated to dryness under vacuum. The crude product is purified by chromatography on alumina, using dichloromethane containing 2% of methanol. The title product is obtained (0.31 g);

Yield: 67%

C₁₃H₁₄N₂: 198.26

¹H NMR (CDCl₃): 0.70 (t, 1H); 1.51 (dd, 1H); 2.89 (dd, 1H); 3.20 (d, 1H); 3.62 (d, 1H); 3.66 (s, 4H); 7.12 (m, 0.2H); 7.17 (m, 114); 7.25 (m, 1H).

Oxalate of the title compound:

mp: 1.64-7166° C.

C₁₅H₁₆N₂O₄: 288.29

Calculated %: C, 62.49; H, 5.59; N, 9.72. Found %: C, 62.46; H, 5.72; N, 9.66.

¹H NMR (DMSOd₆): 0.93 (t, 1H); 2.03 (dd, 1H); 3.24 (d, 1H); 3.29 (dd, 1H); 3.54 (d, 1H); 3.83 (s, 4H); 7.18 (m, 2H); 7.25 (m, 1H); 7.36 (m, 1H).

The compounds of formula (XVII-1) are resolved by chromatographic separation of the diastereoisomeric keto esters (VII-3) of (R)-(−)-pantolactone (RN 599-04-2):

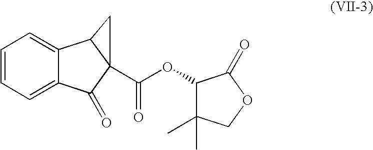

### Example 9

### (+)-2-(1a,6-Dihydro-1H-cyclopropa[a]inden-6a-yl)-4,5-dihydro-1H-imidazole (+)-(XVII-1)

Hydrochloride of the title compound:

mp: 245-247° C.

C₁₃H₁₅CIN₂: 234.73

Calculated %: C, 66.52; H, 6.44; N, 11.93. Found %: C, 66.34; H, 6.65; N, 11.71

¹H NMR (D₂O): 1.01 (t, 1H); 1.92 (dd, 1H); 3.26 (m, 1H); 3.34 (d, 1H); 3.47 (d, 1H); 3.93 (s, 4H); 7.28 (m, 2H); 7.32 (m, 1H); 7.42 (m, 1H).

[α]²⁵_{D}: +218.2° C. (c=0.369, methanol).

### Example 10

### (−)-2-(1a,6-Dihydro-1H-cyclopropa[a]inden-6a-yl)-4,5-dihydro-1H-imidazole (−)-XVII-1)

Hydrochloride of the title compound:

mp: 245-247° C.

C₁₃H₁₅CIN₂: 234.73

Calculated %: C, 66.52; H, 6.44; N, 11.93. Found %: C, 65.85; H, 6.45; N, 11.69.

¹H NMR (D₂O): 1.01 (t, 1H); 1.92 (dd, 1H); 3.26 (m, 1H); 3.34 (d, 1H); 3.47 (d, 1H); 3.93 (s, 0.4H); 7.28 (m, 2H); 7.32 (m, 1H); 7.42 (m, 1H).

[α]²⁵_{D}: −219.90 (c=0.467, methanol).

### Example 11

### 2-(1-exo-Methyl-1a,6-dihydro-1H-cyclopropa-[a]inden-6a-yl)-4,5-dihydro-1H-imidazole (XVII-2)

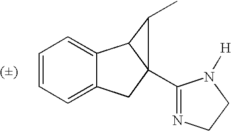

Working as in example 8, but using compound (XVI-2) instead of the compound of formula (XVI-1), the title compound is obtained.

Yield: 30%

C₁₄H₁₆N₂: 212.28

¹H NMR (CDCl₃): 0.88 (m, 1H); 1.22 (d, 3H); 2.57 (d, 1H); 3.19 (d, 1H); 3.35 (d, 1H); 3.63 (s, 4H); 7.10 (m, 3H); 7.25. (d, 1H).

Fumarate of the title compound:

mp: 133-135° C.

C₁₈H₂₀N₂O₄: 328.37

Calculated %: C, 65.84; H, 6.14; N, 8.53. Found %: C, 65.51; H, 6.35; N, 8.65

¹H NMR (DMSOd₆): 1.08 (m, 1H); 1.17 (d, 3H); 3.15 (d, 1H); 3.29 (d, 1H); 3.35 (d, 1H); 3.82 (s, 4H); 6.43. (s, 2H); 7.13 (m, 2H); 7.19 (m, 1H); 7.32 (m, 1H).

### Example 12

### 2-(6-Methyl-1a,6-dihydro-1H-cyclopropa[a]-inden-6a-yl)-4,5-dihydro-1H-imidazole (X)

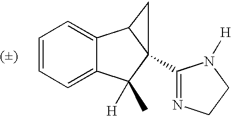

Working as in example 8, but using compound (IX) instead of the compound of formula (XVI-1), the title compound is obtained.

Yield: 68%

C₁₄H₁₆N₂: 212.28

¹H NMR (CDCl₃): 0.65 (t, 1H); 1.40 (d, 3H); 1.63 (dd, 1H); 2.81 (dd, 1H); 3.65 (s, 4H); 3.97 (q, 1H); 7.16 (m, 3H); 7.23 (m, 1H).

Hydrochloride of the title compound:

Sublimation: 250° C.

C₁₄H₁₇CIN₂: 248.76

Calculated %: C, 67.60; H, 6.89; N, 11.26. Found %: C, 67.18; H, 6.96; N, 11.04.

¹H NMR (D₂O): 0.97 (t, 1H); 1.38 (d, 3H); 1.75 (dd, 1H); 3.15 (dd, 1H); 3.82 (q, 1H); 3.92 (s, 4H); 7.27 (m, 3H); 7.31 (m, 1H).

### Example 13

### 6a-(4,5-Dihydro-1H-imidazol-2-yl)-1,1a,6,6a-tetrahydrocyclopropa[a]inden-6-β-ol (XII-1)

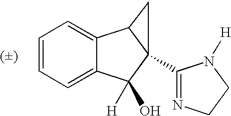

Working as in example 8, but using compound (XI-1) instead of the compound of formula (XVI-1), the title compound is obtained.

Yield: 27%

C₁₃H₁₄N₂O: 214.26

¹H NMR (DMSOd₆): 1.29 (t, 1H); 1.85 (dd, 1H); 3.19 (dd, 1H); 3.81 (s, 4H); 5.76 (s, 1H); 7.22 (m, 2H); 7.28 (m⁻²H).

Hydrochloride of the title compound:

mp: 229-231° C.

C₁₃H₁₅CIN₂O: 250.73

Calculated %: C, 62.28; H, 6.03; N, 11.17. Found %: C, 62.36; H, 6.05; N, 11.29.

¹H NMR (D₂O): 1.41 (t, 1H); 1.84 (dd, 1H); 3.30 (dd, 1H); 3.97 (s, 4H); 5.85 (s, 1H); 7.36 (m, 4H).

### Example 14

### 2-(6-Methoxy-1a,6-dihydro-1H-cyclopropa[a]-inden-6a-yl)-4,5-dihydro-1H-imidazole (XV-1)

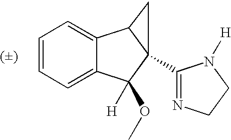

Working as in example 8, but using compound (XIV-1) instead of the compound of formula (XVI-1), the title compound is obtained.

Yield: 51%

C₄H₁₆N₂O: 228.28

¹H NMR (CDCl₃): 1.25 (t, 1H); 1.66 (dd, 1H); 2.75 (dd, 1H); 3.59 (s, 3H); 3.64 (s, 4H); 5.61 (s, 1H); 7.18 (m, 3H); 7.27 (m, 1H).

Fumarate of the title compound:

mp: 173-175° C.

C₁₈H₂₀N₂O₅: 344.37

Calculated %: C, 62.78; H, 5.85; N, 8.13. Found %: C, 62.64; H, 5.94; N, 8.15.

¹H NMR (DMSOd₆): 1.18 (t, 1H); 2.09 (dd, 1H); 3.08 (dd, 1H); 3.44 (s, 3H); 3.75 (s, 4H); 5.68 (s, 1H); 6.44 (s, 2H); 7.17-7.31 (m, 4H).

The resolution is performed by chromatographic separation on a chiral support (Chiralpack AD, eluent: hexane containing 5% methanol).

### Example 15

### (+)-2-(6-Methoxy-1a,6-dihydro-1H-cyclopropa[a]inden-6a-yl)-4,5-dihydro-1H-imidazole (+)-(XV-1)

Fumarate of the title compound:

mp: 170-172° C.

C₁₈H₂₀N₂O₅: 344.37

Calculated %: C, 62.78; H, 5.85; N, 8.13. Found %: C, 62.55; H, 5.89; N, 8.05.

¹H NMR (DMSOd₆): 1.19 (t, 1H); 2.09 (dd, 1H); 3.09 (dd, 1H); 3.44 (s, 3H); 3.75 (s, 4H); 5.67 (s, 1H); 6.44 (s, 2H); 7.19-7.31 (m, 0.4H)

[α]²⁵_{D}: +174.150 (c 0.16, methanol).

### Example 16

### (−)-2-(6-Methoxy-1a,6-dihydro-1H-cyclopropa[a]inden-6a-yl)-4,5-dihydro-1H-imidazole (−)—(XV-1)

Fumarate of the title compound:

mp: 170-172° C.

C₁₈H₂₀N₂O₅: 344.37

Calculated %: C, 62.78; H, 5.85; N, 8.13. Found %: C, 62.45; H, 5.92; N, 8.05.

¹H-NMR (DMSOd₆): 1.19 (t, 1H); 2.09 (dd, 1H); 3.09 (dd, 1H); 3.44 (s, 3H); 3.75 (s, 4H); 5.67 (s, 1H); 6.44 (s, 2H); 7.19-7.31 (m, 4H)

[α]²⁵_{D}: −164.16°. (c=0.14, methanol).

### Example 17

### 2-(2,6-Dimethoxy-1a,6-dihydro-1H-cyclopropa[a]inden-6a-yl)-4,5-dihydro-1H-imidazole (XV-1a)

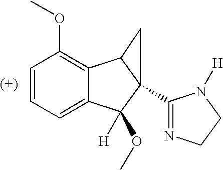 Working as in example 8, but using compound (XIV-1a) instead of the compound of formula (XVI-1), the title compound is obtained.

Yield: 55%

C₁₅H₁₈N₂O₂: 258.31

¹H NMR (CDCl₃): 1.31 (t, 1H); 1.68 (dd, 1H); 2.84 (dd, 1H); 3.58 (s, 3H); 3.63 (s, broad, 4H); 3.83 (s, 3H); 5.68 (s, 1H); 6.73 (d, 1H); 6.90 (d, 1H); 7.14 (t, 1H).

Fumarate of the title compound:

mp: 177-179° C.

C₁₉H₂₂N₂O₆: 374.39

Calculated %: C, 60.95; H, 5.92; N, 7.48. Found %: C, 60.24; H, 6.39; N, 7.08.

¹H NMR (DMSOd₆): 1.19 (t, 1H); 2.03 (dd, 1H); 3.05 (dd, 1H); 3.43 (s, 3H); 3.74 (s, 4H); 3.80 (s, 3H); 5.64 (s, 1H); 6.45 (s, 2H); 6.86 (m, 2H); 7.19 (t, 1H).

### Example 18

### 2-(3-Fluoro-6-methoxy-1a,6-dihydro-1H-cyclopropa[a]inden-6a-yl)-4,5-dihydro-1H-imidazole (XV-1b)

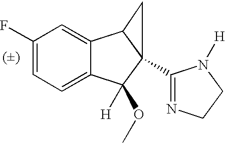 Working as in example 8, but using compound (XIV-1b) instead of the compound of formula (XVI-1), the title compound is obtained.

### Example 19

### 2-(3-Fluoro-1-exo-methyl-1a,6-dihydro-1H-cyclopropa[a]inden-6a-yl)-4,5-dihydro-1H-imidazole (XVII-2b)

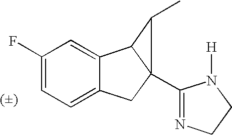 Working as in example 8, but using compound (XIV-2) instead of the compound of formula (XVI-1), the title compound is obtained.

The compounds of formula (1) and the therapeutically acceptable salts thereof have advantageous pharmacological properties.

The results of the tests are collated in the following table:

| | | | | Scopolamine- |
|---|---|---|---|---|
| | | | | induced memory |
| | | | Intrinsic | deficiency |
| | | | activity | amplitude of |
| | Affinity (pKi) | | % | the effect % |
| Compound | Alpha-2A | Alpha-2B | stimulated | (dose, mg/kg i.p.) |
| XV-1 | 7.9 | 6.5 | −2 | +168 (2.5) |
| (+) -XV-1 | 8.4 | <5 | −63 | — |
| XVII-2 | 8.5 | 7.7 | −64 | +181 (0.63) |
| (−) — | — | — | +100 | — |
| adrenalin | | | | |
| donepezil | — | — | — | +67 (0.16) |

Scopolamine- Intrinsic induced Compound Affinity (pKi)-activity memory deficiency amplitude of % the effect % Alpha-2A Alpha-2B stimulated (dose, mg/kg . . . _{—}_i.p.)—-XV-.l 7.9 6.5- -2+168 (2.5) (+)—XV-1 8.4<5-63 _XVII-2 8.5 7.7-64+181 (0.63) * (−)- - -: . _‘+’, 100 adrenalin donepezil _+67 (0.16)
Binding to the Adrenergic Alpha-2 Receptors:

Membranes of C6 cells permanently expressing either the human alpha-2A or alpha-2B receptor are prepared in Tris-HCl (pH=7.6). The binding tests are performed with 2 nM [³H]RX 821002. The incubation medium is composed of 0.4 ml of cell membranes (10 μg of proteins), 0.05 ml of radioligand and 0.05 ml of test product or of phentolamine (10 μM) to determine the nonspecific binding. The reaction is quenched after incubation for 30 minutes at 25° C. by adding 3 ml of cold 50 mM Tris-HCl (pH=7.6), followed by filtration through Whatman GF/B filters using a Brandel. The Ki values are calculated according to the equation Ki=IC₅₀/(1+C/Kd) in which C is the concentration and Kd the dissociation constant, pKi=−logKi. Under these conditions, it is seen that the compounds of the invention have high affinity for the receptors of the human adrenergic alpha-2A subtype, whereas they have little or no affinity for the receptors of the human adrenergic alpha-2B subtype (cf. above table). This unexpected receptor selectivity of the compounds of the invention, compared with the compounds of the prior art, may have a favorable impact on their tolerance.

Measurement of the Activation of the Adrenergic Alpha-2 Receptors:

The responses as GTPγS are performed on membrane preparations in 20 mM HEPES (pH=7.4) with 30 μM of GDP, 100 mM of NaCl, 3 mM of MgCl₂ and 0.2 mM of ascorbic acid. The maximum stimulation of the GTPγS is determined in the presence of 10 mM of (−)-adrenalin and calculated versus the basal GTPγS response. The results are expressed versus either adrenalin or RX 81105-9. Under these conditions, the compounds of the invention are distinguished from most of the compounds of the prior art of the 4,5-dihydro-1H-imidazole and/or 1H-imidazole type in that they behave rather as inverse agonists on human alpha-2A adrenergic receptors (cf. above table).

Test of Scopolamine-Induced Memory Deficiency:

Scopolamine has amnesiant properties in man and animals. Thus, its administration to a healthy person causes certain symptoms similar to what is observed in Alzheimer's disease. Scopolamine-induced memory deficiency is thus used as an experimental pharmacological model of this pathology. Scopolamine reduces the capacity for acquisition, memorization and recall in a test of passive avoidance in rats. This involves measuring the reticence, after learning, that an animal shows with regard to entering a dark compartment where it receives a mild electric shock. The administration of scopolamine suppresses this reticence, and the test compounds counter the effect of scopolamine. The experimental protocol used is described in Psychopharmacol. 1992, 106, 26-30.

The compounds of the invention show considerable activity in this test (cf. above table). The amplitude of the effect obtained with the compounds of the invention is higher than that, for example, of donezepil, an acetylcholinesterase inhibitor used clinically for the treatment of Alzheimer's disease (chem. Rec. 2001, 1(1), 63-73). The compounds of the invention are thus capable of efficiently countering scopolamine-induced memory deficiency.

The results of the tests thus show that the compounds of formula (1):
have high affinity for the human adrenergic receptors of the alpha-2A subtype;
have little or no affinity for the human alpha-2B adrenergic receptors;
generally behave like inverse agonists on the human alpha-2A adrenergic receptors;
are active, in vivo, in an animal model considered as representative of the memory disorders manifested during Alzheimer's disease.

As a result, the compounds of the invention and the therapeutically acceptable salts thereof are potentially useful as medicinal products, in particular in the treatment of certain progressive neurodegenerative pathologies, for instance Alzheimer's disease.

The administration of the compounds of the invention may be performed via the oral, nasal, sublingual, rectal or parenteral route. A preparation of the compounds of the invention is given hereinbelow as a nonlimiting formulation example. The ingredients and other therapeutically acceptable ingredients may be introduced in other proportions without modifying the scope of, the invention. The term “active ingredient”used in the formulation example hereinbelow refers to a compound of formula (1) or an addition salt or possibly a hydrate of an addition salt of the compound of formula (1) with a pharmaceutically acceptable mineral acid or organic acid.

Example of a Pharmaceutical Composition

Preparation formulation for 1000 tablets each containing 10 mg of the active ingredient:

| | |
|---|---|
| Active ingredient | 10 g |
| Lactose | 100 g |
| Wheat starch | 10 g |
| Magnesium stearate | 3 g |
| Talc | 3 g |

1. A compound of general formula (1): 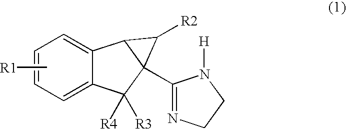
in which:
(a) R1 represents a hydrogen atom, a fluorine atom or a methoxyl (OCH₃) group and the substituent R1 on the aromatic carbocycle may be in position 2, 3, 4 or 5;
(b) R2 represents a hydrogen atom or a methyl group;
(c) R3 represents a hydrogen atom, a methyl group, a hydroxyl group (OH) or a methoxyl group (OCH₃);
(d) R4 is a hydrogen atom; or
(e) R3 and R4 together represent a carbonyl group (C═O);
or a salt or hydrate of said salt with a pharmaceutically acceptable mineral acid or organic acid, or the tautomeric or isomeric forms thereof.

2. The compound of claim 1, selected from the group consisting of:
2-(1a,6-dihydro-1H-cyclopropa[a]inden-6a-yl)-4,5-dihydro-1H-imidazole;
2-(1-methyl-1a,6-dihydro-1H-cyclopropa[a]inden-6a-yl)-4,5-dihydro-1H-imidazole;
2-(2-fluoro-1-methyl-1a,6-dihydro-1H-cyclopropa[a]inden-6a-yl)-4,5-dihydro-1H-imidazole;
2-(3-fluoro-1-methyl-1a,6-dihydro-1H-cyclopropa[a]inden-6a-yl)-4,5-dihydro-1H-imidazole;
2-(4-fluoro-1-methyl-1a,6-dihydro-1H-cyclopropa[a]inden-6a-yl)-4,5-dihydro-1H-imidazole;
2-(5-fluoro-1-methyl-1a,6-dihydro-1H-cyclopropa[a]inden-6a-yl)-4,5-dihydro-1H-imidazole;
2-(6-methyl-1a,6-dihydro-1H-cyclopropa[a]inden-6a-yl)-4,5-dihydro-1H-imidazole;
6a-(4,5-dihydro-1H-imidazol-2-yl)-1a,6a-dihydro-1H-cyclopropa[a]inden-6-one;
6a-(4,5-dihydro-1H-imidazol-2-yl)-1,1a,6,6a-tetrahydrocyclopropa[a]inden-6-ol;
2-(6-methoxy-1a,6-dihydro-1H-cyclopropa[a]inden-6a-yl)-4,5-dihydro-1H-imidazole;
2-(2,6-dimethoxy-1a,6-dihydro-1H-cyclopropa[a]inden-6a-yl)-4,5-dihydro-1H-imidazole;
2-(2-fluoro-6-methoxy-1a,6-dihydro-1H-cyclopropa[a]inden-6a-yl)-4,5-dihydro-1H-imidazole;
2-(3-fluoro-6-methoxy-1a,6-dihydro-1H-cyclopropa[a]inden-6a-yl)-4,5-dihydro-1H-imidazole;
2-(4-fluoro-6-methoxy-1a,6-dihydro-1H-cyclopropa[a]inden-6a-yl)-4,5-dihydro-1H-imidazole; and
2-(5-fluoro-6-methoxy-1a,6-dihydro-1H-cyclopropa[a]inden-6a-yl)-4,5-dihydro-1H-imidazole;
or a salt or hydrate of said salt with a pharmaceutically acceptable mineral acid or organic acid, or the tautomeric and isomeric forms of each said compound.

3. A process for preparing a compound according to claim 1 comprising reacting a compound of formula (VII): 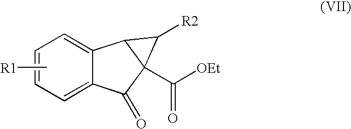
in which R1 and R2 have the same meaning as in formula (1), with:
(a) methyltriphenylphosphonium bromide in the presence of a base, and the compound formed is reduced with diimide and the reduced compound formed is treated with ethylenediamine to form the compound of formula (X), 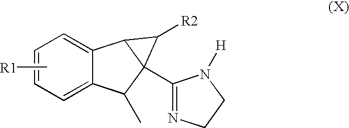
which R1 has the same meaning as in formula (1) and R2 is a hydrogen atom or a methyl group; or
(b) a reducing agent, and the compound formed is treated with ethylenediamine to form the compound of formula (XII), 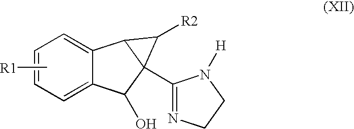
in which R1 and R2 have the same meaning as in formula (1); and, optionally,
further oxidizing the compound of formula (XII) to form the compound of formula (XIII), 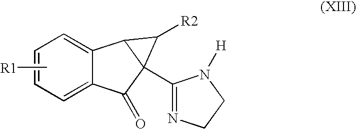
in which R1 and R2 have the same meaning as in formula (1); or
(c) a reducing agent, and the compound formed is methylated in the presence of methyl iodide and a silver salt, and the product formed is then treated with ethylenediamine to form the compound of formula (XV), 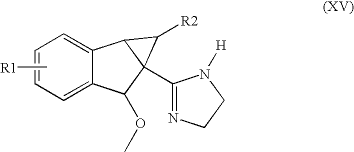
in which R1 and R2 have the same meaning as in formula (1); or
(d) a reducing agent in strong acid medium, and the compound formed is treated with ethylenediamine to form the compound of formula (XVII), 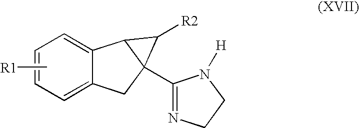
in which R1 and R2 have the same meaning as in formula (1).

4. A compound of general formula (VII): 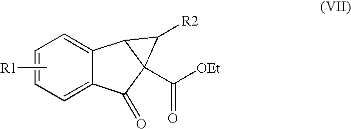
in which R1 and R2 have the same meaning as in formula (1).

5. A process for preparing a compound of formula (VII), 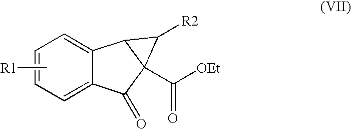
comprising intramolecular cyclization reaction of a compound of formula (VI) 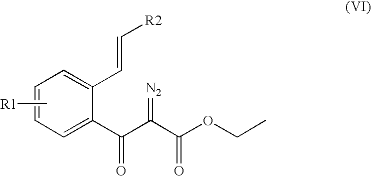
in which R1 represents a hydrogen atom, a fluorine atom or a methoxyl (OCH₃) group and the substituent R1 on the aromatic carbocycle may be in position 2, 3, 4 or 5; and R2 represents a hydrogen atom or a methyl group.

6. The compound of claim 1, wherein the substituents R2 and 4,5-dihydro-1H-imidazole are in syn-periplanar positions relative to the plane defined by the cyclopropane ring, R1, R3 and R4 have the same meaning as in formula (1) and R2 is a methyl group (CH₃).

7. The compound of claim 1, wherein R3 and 4,5-dihydro-1H-imidazole are in anti-periplanar positions relative to the plane defined by the indane ring, R1 and R2 have the same meaning as in formula (1), R3 represents a methyl group, a hydroxyl group (OH) or a methoxyl group (OCH₃) and R4 is a hydrogen atom.

8. The compound of claim 1 in which said compound is in levorotatory or dextrorotatory enantiomeric form.

9. A medicament comprising a compound as claimed in claim 1.

10. A pharmaceutical composition, comprising as active ingredient, a compound as claimed in claim 1 and an inert or pharmaceutically acceptable carrier and optionally comprising at least one additional active medicament.

11. (canceled)

12. (canceled)

13. A method of treating Alzheimer's disease, or the evolution of Alzheimer's disease, said treatment comprising administering to a patient in need of such treatment a pharmaceutical composition comprising a therapeutically effective amount of a compound of claim 1.

14. A method of treating Parkinson's disease or Creutzfeld-Jacob disease or strokes, or the evolution of Parkinson's disease or Creutzfeld-Jacob disease or strokes, said treatment comprising administering to a patient in need of such treatment a pharmaceutical composition comprising therapeutically effective amount of a compound of claim 1.

15. Tautomeric and isomeric forms of a compound of claim 1.

16. A compound of claim 15 wherein said isomeric forms include enantiomers, mixtures of enantiomers, stereoisomers, racemates and mixtures thereof.
